(19) United States
(12) Patent Application Publication
(10) Pub. No.: US 2019/0009042 A1
(43) Pub. Date: Jan. 10, 2019

(54) CUFF INFLATORS, INDICATORS AND ASSEMBLIES

(71) Applicant: SMITHS MEDICAL INTERNATIONAL LIMITED, Ashford (GB)

(72) Inventors: Timothy Bateman, Hythe (GB); Stephen James Field, Canterbury (GB); Andrew Thomas Jeffrey, Hythe (GB); Christopher John Woosnam, Great Sutton (GB)

(73) Assignee: SMITHS MEDICAL INTERNATIONAL LIMITED, Ashford (GB)

(21) Appl. No.: 16/067,385

(22) PCT Filed: Dec. 20, 2016
(86) PCT No.: PCT/GB2016/000218

§ 371 Date: Jun. 29, 2018

(30) Foreign Application Priority Data
Jan. 23, 2016 (GB) 1601396.3

(51) Int. Cl. A61M 16/04; A61M 16/00

(52) U.S. Cl. CPC A61M 16/044; A61M 2016/0027; A61M 16/0003

(57) ABSTRACT

A tracheal tube cuff inflator/indicator has a coloured pressure flag (211) slidable against the action of a spring (212) along the inside of a transparent housing (210) by change of pressure. The flag has three coloured regions along its length. A sleeve (310) with a transparent central window (311) is slidable frictionally along the outside of the housing so that the window can be aligned with a central green region (211G) when correct pressure is achieved in the cuff (10). Any change in pressure of the cuff (10) moves the flag (211) so that the green portion (211G) moves away from the window (311) in the outer sleeve, thereby indicating to the user that the cuff pressure has changed. The inflator/indicator also has an elastomeric balloon (220) acting as a pressure reservoir that can be manually compressed if it is necessary to increase pressure in the cuff.

This invention relates to cuff pressure indicators of the kind including a housing adapted for connection with the interior of a sealing cuff and an indicator member within the housing and movable relative to the housing by pressure change in the cuff.

Tracheal tubes are used to supply ventilation and anesthetic gases to a patient, such as during surgery. The tracheal tube may be inserted via the mouth or nose, in the case of an endotracheal tube, or may be inserted via a surgically-made tracheostomy opening in the neck, in the case of a tracheostomy tube. Most, but not all, tracheal tubes have some form of a seal on their outside which forms a seal between the outside of the tube and the inside of the trachea so that gas flow is confined to the bore of the tube and cannot flow around the outside of the tube, between the tube and the trachea.

The most common form of seal is provided by an inflatable cuff that is inflated and deflated via a small bore lumen extending along the tube and connected towards its rear end to an inflation line terminated by an inflation indicator, valve and connector. These inflatable cuffs may be of the high-volume/low-pressure kind where the cuff is formed of a flexible plastics material moulded with a natural annular or doughnut shape that is inflated without stretching, to contact the wall of the trachea, by relatively low-pressure gas supplied via the inflation line. Alternatively, the cuff may be of the low-volume/high-pressure kind where the cuff is of an elastic material that lies close to the tube shaft when uninflated but is inflated and stretched to a larger diameter by relatively high pressure gas or liquid supplied via the inflation line. One problem with these cuffs is the difficulty of preventing secretions that collect above the cuff leaking between the cuff and the trachea and entering the bronchial passages. The leakage of such secretions is thought to contribute to ventilator-associated pneumonia (YAP). It is particularly important that the cuff be inflated to the correct pressure since too low a pressure could allow leakage past the cuff and too high a pressure could cause damage to the tracheal lining. In an attempt to ensure that the cuff is inflated correctly the clinician may inflate it to a known pressure (typically between 20 and 30 cm Hg) using an inflation device with a pressure gauge attached. Alternatively, the clinicians may inflate the cuff while listening for the sound of exhaled air passing around the outside of the cuff before it is fully inflated. When the cuff is correctly inflated there will be no passage of air between the outside of the cuff and the trachea so these sounds will cease. Although the cuff may be correctly inflated initially, the seal with the trachea may vary during use. One reason for this is that the patient or the tracheal tube may be moved causing the cuff to be distorted or moved, thereby affecting the seal. Pressure in the cuff may also change because of permeation of anesthetic gases through the cuff wall. Cuffs of certain materials can also allow water vapour to permeate the cuff and increase pressure within the cuff. The cuff will also lose pressure over time as gas permeates gradually through the wall of the cuff from the higher pressure in the cuff to the lower ambient pressure outside the cuff. If the seal provided by the cuff becomes less effective over time it may be necessary to reinflate the cuff. This can be a problem because an inflation syringe might not be readily available.

Various suggestions have been made how to indicate and control the pressure within such cuffs. The PressureEasy® device from Smiths Medical is connected in line with the sealing cuff and includes a coloured flag that is displaced by pressure in the cuff to indicate whether pressure is in the correct range. This device is connected to the ventilation circuit and is arranged to increase cuff pressure when ventilation pressure increases. U.S. Pat. No. 4,501,273 describes an endotracheal tube with a sealing cuff connected to a constant pressure device in the form of an inflatable member within a rigid housing.

It is an object of the present invention to provide an alternative cuff inflator, indicator or assembly.

According to one aspect of the present invention there is provided a cuff pressure indicator of the above-specified kind, characterised in that the indicator includes a manually-displaceable member having a portion that can be aligned with the indicator member when the cuff is at the desired pressure so that change in cuff pressure is indicated by movement of the indicator member away from the portion of the manually-displaceable member.

The portion of the manually-displaceable member may be a window through which the indicator member can be viewed. The manually-displaceable member may be movable along the length of a part of the housing. The manually-displaceable member is preferably a tubular member slidable along the part of the housing. The indicator member may be movable within the housing against the action of a resilient member. The indicator member may be divided into three differently-marked regions, such as of different colours.

According to another aspect of the present invention there is provided a cuff inflator including an expansible member having a fluid inlet and a fluid outlet adapted for connection to a sealing cuff such that, the expansible member is expanded when fluid is supplied to the inflator, characterised in that the expansible member is manually compressible, and that the inflator includes a valve between the outlet of the expansible member and the cuff that is normally closed but can be opened by compressing the expansible member to enable additional fluid to be supplied to the cuff.

The expansible member may be an elastomeric bulb. The outlet of the cuff inflator may be connected with a cuff pressure indicator according to the above one aspect of the present invention.

According to a further aspect of the present invention there is provided an assembly of a medico-surgical tube including an inflatable sealing cuff and a cuff pressure indicator according to the above one aspect of the present invention, wherein the sealing cuff is connected to the cuff pressure indicator such that the cuff pressure indicator provides an indication of pressure change within the sealing cuff.

According to a fourth aspect of the present invention there is provided an assembly of a medico-surgical tube including an inflatable sealing cuff and a cuff inflator according to the above other aspect of the present invention, wherein the sealing cuff is connected to the cuff inflator such that pressure in the sealing cuff can be increased by manually compressing the expansible member in the cuff inflator.

An assembly of a cuffed endotracheal tube and a combined cuff pressure indicator and inflator according to the present invention will now be described, by way of example, with reference to the accompanying drawings, in which:

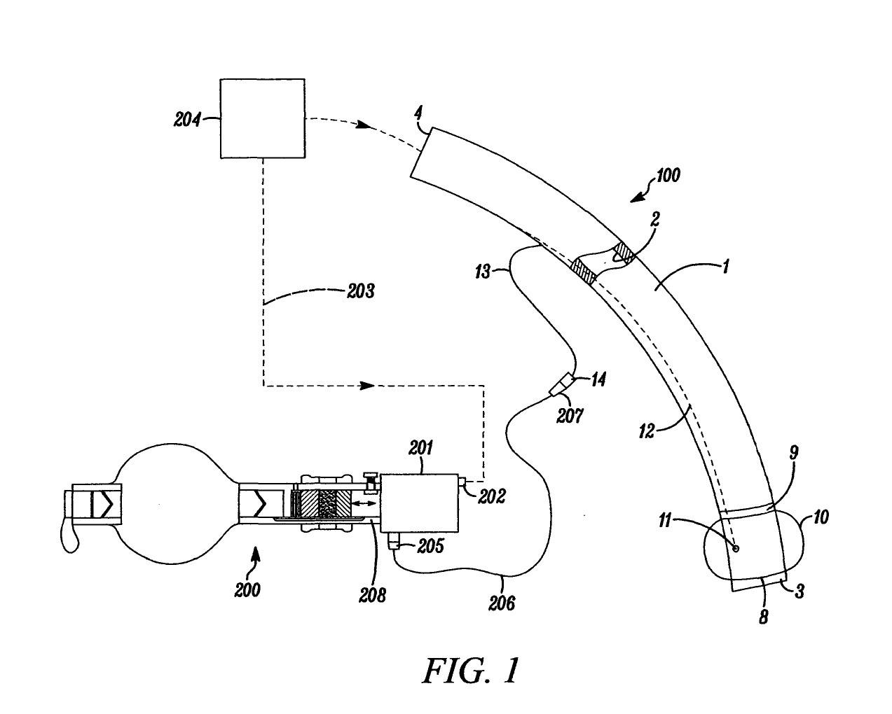

FIG. 1 is a side elevation view of the assembly; and

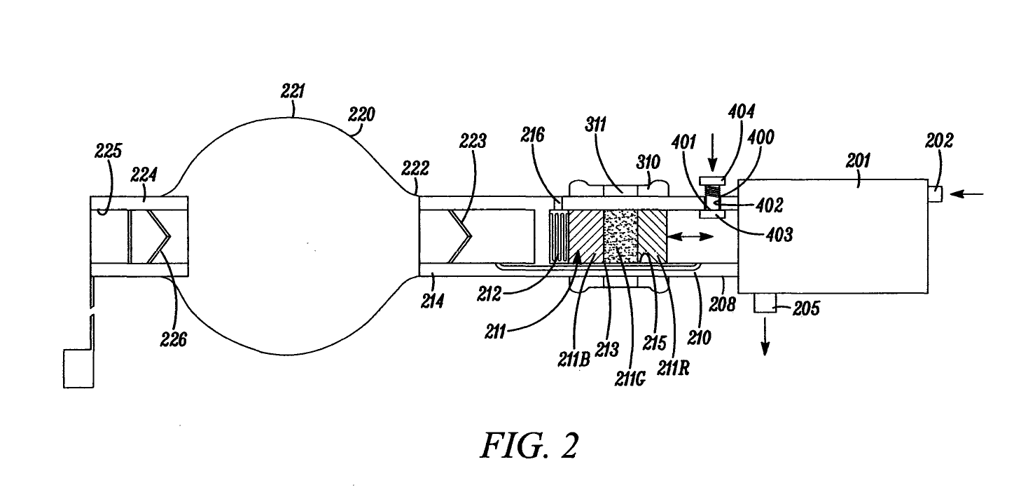

FIG. 2 is an enlarged cross-sectional side elevation of the cuff pressure indicator and inflator.

The assembly includes an endotracheal tube 100 and a combined cuff pressure indicator/inflator 200 connected to the tube. The endotracheal tube 100 includes a tubular shaft 1 having a bore 2 extending along its length. The tube shaft 1 is curved along its length from its patient end 3 to its machine end 4. The shaft 1 is extruded or moulded from a plastics material such as PVC. Towards its patient end 3 the tube 100 has sealing means provided by an inflatable cuff 10 embracing the shaft 1. The cuff 10 is of the high-volume/low-pressure kind so that it has a relatively floppy shape when deflated but, when inflated, it fills out at low pressure to a diameter matching the internal diameter of the trachea. The cuff 10 is attached to the shaft 1 by two collar portions 8 and 9 at opposite ends. The cuff 10 extends over an opening 11 on the outer surface of the shaft 1 into an inflation lumen 12 extending along the shaft within its wall thickness. The inflation lumen 12 is connected towards the rear end of the tube with a small-bore inflation line 13 terminated by a connector 14.

The combined cuff pressure indicator/inflator 200 includes an optional pressure controller 201 such as of the kind available from Smiths Medical under the trade mark PressureEasy. The controller 201 has a cylindrical chamber with a first inlet 202 connected by a gas line 203 to a ventilation circuit 204 so that the chamber receives a sample of the same gas supplied to the machine end of the endotracheal tube 100. The indicator/inflator 200 also has an outlet 205 connected via an inflation line 206 extending to a connector 207 coupled with the connector 14 at the end of the inflation line 13. The chamber of the controller 201 contains a displaceable member acted on by pressure at the first inlet 202 to move it towards the outlet 205 and thereby increase pressure at the outlet when pressure at the inlet increases. Further information about operation of such controllers can be found in U.S. Pat. No. 4,501,273 and US2012090619 although details of these are not needed for an understanding of the present invention. The assembly need not include a pressure controller 201 responsive to ventilation pressure; instead, the inflator/indicator 200 could be connected directly with the tube 100.

The indicator/inflator 200 has an axially arranged inlet 208 by which gas is supplied to the indicator/inflator for inflating the sealing cuff 10. The inlet 208 takes the form of a tubular, transparent housing 210 of cylindrical shape. The housing 210 contains a slidable indicator member or flag 211 of cylindrical shape that is displaceable sealingly along the inside of the housing. The outside of the indicator flag 211 is divided into three regions along the flag marked with different colours, namely a central region 211G, coloured green, a left-hand region 211B towards the closed end of the chamber 215, coloured blue, and a right-hand region 211R away from the closed end of the chamber, coloured red. It will be appreciated that the indicator flag could have more or fewer regions with different markings. The indicator could be the same colour along its length, correct inflation being indicated by whether or not the indicator was visible. The indicator flag 211 is urged forwardly along the housing 210 by a resilient member in the form of a helical spring 212 located at a closed end of a cylindrical chamber 215 within the housing 210. The closed end of the chamber 215 has a vent opening 216 to prevent pressure build up when the flag 211 is moved towards the closed end. The housing 210 also has a bypass channel 213 extending along it so that gas can flow along the housing from the inlet end 214 to the inlet 208 of the chamber 201. The outside of the housing 210 supports a manually-displaceable, slidable tubular marker member or sleeve 310 of cylindrical shape that is a close, frictional, sliding fit along the housing and remains in position until manually displaced. The outside of the housing 210 and the sleeve 310 could have cooperating detent features or the like so that the sleeve can be clicked into a series of closely-spaced positions along the housing. The marker sleeve 310 is mainly of an opaque material but has a central annular region 311 that is transparent, providing a narrow window through which a part of the length of the indicator flag 211 can be viewed, equivalent to one of the three regions. The window 311 may be of a transparent material or it may be provided by an aperture in the sleeve 310. The inlet end 214 of the housing 210 connects with an expansible member in the form of a manually-compressible elastic bulb 220 having a generally spherical wall 221 of a thin elastomeric material that is exposed externally for manual actuation. The bulb 220 opens at its outlet end 222 via a one-way valve 223 into the inlet end of the housing 210, which opens to allow flow from the bulb 220 into the housing but prevents flow in the opposite direction. Bulbs of other shapes could be used. At its opposite end the bulb 220 includes a capped inlet 224 with a female luer opening 225 and a one-way valve 226, which opens to allow flow from the inlet into the bulb and prevents flow in the opposite direction. Alternative expansible members could be used, such as compressible bellows or the like.

The indicator/inflator 200 also includes a deflate valve 400 at a location between the chamber 201 and the bulb 220 along the housing 210. The deflate valve 400 has a spring-loaded valve member 401 extending through an aperture 402 in the wall of the housing 210 the valve member has a seal 403 that normally blocks the aperture and prevents air flow out through the aperture. When it is necessary to reduce pressure in the cuff 10, such as to reduce it to a desired level or to deflate it completely before removing the tube 100, a button 404 on the outer end of the valve member 401 is pushed in so that the seal 403 is pushed down away from the aperture 402 to allow air to escape through the aperture out from the housing 210.

The endotracheal tube 100 is inserted in the trachea with the cuff 10 deflated. Once positioned correctly the cuff 10 is inflated using a cuff inflation device or syringe or the like filled with air. Alternatively, the bulb 220 itself could be used to inflate the cuff 10 initially. Where a syringe is used, the inlet 224 of the indicator/inflator 200 is uncapped and the nose of the syringe (not shown) is inserted in the luer opening 225. The plunger of the syringe is pressed slowly along its barrel so that air is supplied via the valve 226 to the interior of the bulb 220. The size of the bulb 220 is chosen so that it is stretched by the air delivered by the syringe and a volume of air is contained in the bulb under pressure. The raised pressure in the bulb 220 opens the valve 223 at its outlet end so that air also flows via the bypass channel 213 along the housing 210. Air flows from the housing 210 into the chamber 201 from where it flows via the outlet 205 to the inflation lines 206 and 13. Air flowing along the inflation lines 206 and 13 flows to the inflation lumen 12 along the tube shaft 1, thereby inflating the sealing cuff 10. At the same time the right-hand end of the indicator flag 211 is exposed to the pressure in the housing 210. This causes the indicator 211 to be displaced to the left against the force of the helical spring 212, the extent of displacement being proportional to the pressure in the housing 210 and hence to the pressure in the sealing cuff 10. The clinician continues to supply air to the bulb 220 and hence to the sealing cuff 10 until the cuff achieves the desired seal with the trachea. This is typically detected by listening to the hissing sound made by flow of gas around the outside of the cuff as the patient exhales until the sound ceases, indicating that an effective seal has been achieved. The clinician then removes the syringe and recaps the inlet 224 of the bulb 220 closed. The position of the indicator 211 along the housing 210 therefore indicates that the correct pressure has been achieved. The clinician then marks this position by sliding the marker member 310 to the left or right so that the window 311 aligns with the central green portion 211G of the indicator flag 211. In this way, any change in pressure can easily be observed if the green portion of the indicator flag 211 should move away from the transparent window 311. If the right-hand portion 211R is moved within the window 311 by an excessive pressure a red colour becomes visible to indicate that pressure in the cuff is too high. If, however, the indicator flag 211 should be moved to the right because of a fall in pressure, the left-hand portion 211B of the indicator would become visible in the window 311 so that this appears blue.

After insertion, if the clinician should subsequently notice that the cuff pressure is too low (a blue window) he simply squeezes the bulb 220 gently to open the one-way valve 223 at the bulb's outlet and deliver an additional small volume of gas to the cuff 10 until the window appears green again. This avoids the need to find a separate inflation device such as a syringe since the bulb 220 is always attached. If, however, the cuff pressure should rise above the desired level (showing a red window) the clinician simply opens the deflate valve 400 briefly to allow some air to escape to return the indicator to the green colour.

It is not essential for the displaceable indicator member to be movable along its length by a pressure change since alternative indicators could, for example, be rotated by the pressure change. The cuff inflation indicator/inflator of the present invention need not be used with a device for adjusting cuff pressure automatically in response to change in ventilation pressure; instead it could be connected directly to the tube inflation line.

Instead of using air as the inflation fluid it would be possible to use other gases or liquids, such as saline. Instead of a high-volume/low-pressure cuff, the cuff could be of a high-pressure elastic kind that is expanded and stretched by gas pressure supplied to the cuff.

The invention is not confined to endotracheal tubes but could be used on other cuffed medico-surgical tubes.

1-12. (canceled)

13. A cuff pressure indicator including a housing adapted for connection with the interior of a sealing cuff and an indicator member within the housing and movable relative to the housing by pressure change in the cuff, characterised in that the indicator includes a manually-displaceable member having a portion that can be aligned with the indicator member when the cuff is at the desired pressure so that change in cuff pressure is indicated by movement of the indicator member away from the portion of the manually-displaceable member.

14. A cuff pressure indicator according to claim 13, characterised in that the portion of the manually-displaceable member is a window through which the indicator member can be viewed.

15. A cuff pressure indicator according to claim 13, characterised in that the manually-displaceable member is movable along the length of a part of the housing.

16. A cuff pressure indicator according to claim 15, characterised in that the manually-displaceable member is a tubular member slidable along the part of the housing.

17. A cuff pressure indicator according to claim 13, characterised in that the indicator member is movable within the housing against the action of a resilient member.

18. A cuff pressure indicator according to claim 13, characterised in that the indicator member is divided into three differently-marked regions.

19. A cuff pressure indicator according to claim 18, characterised in that the differently marked regions are of different colours.

20. A cuff inflator including an expansible member having a fluid inlet and a fluid outlet adapted for connection to a sealing cuff such that, the expansible member is expanded when fluid is supplied to the inflator, characterised in that the expansible member is manually compressible, and that the inflator includes a valve between the outlet of the expansible member and the cuff that is normally closed but can be opened by compressing the expansible member to enable additional fluid to be supplied to the cuff.

21. A cuff inflator according to claim 20, characterised in that the expansible member is an elastomeric bulb.

22. A cuff inflator according to claim 20, characterised in that the outlet of the cuff inflator is connected with a cuff pressure indicator that includes a housing adapted for connection with the interior of a sealing cuff and an indicator member within the housing and movable relative to the housing by pressure change in the cuff, wherein the indicator includes a manually-displaceable member having a portion that can be aligned with the indicator member when the cuff is at the desired pressure so that change in cuff pressure is indicated by movement of the indicator member away from the portion of the manually-displaceable member.

23. A cuff inflator according to claim 20, characterised in that the cuff inflator is a part of an assembly of a medico-surgical tube that includes an inflatable sealing cuff, wherein the sealing cuff is connected to the cuff inflator such that pressure in the sealing cuff can be increased by manually compressing the expansible member in the cuff inflator.

24. An assembly of a medico-surgical tube comprising an inflatable sealing cuff and a cuff pressure indicator having a housing adapted for connection with the interior of a sealing cuff and an indicator member within the housing and movable relative to the housing by pressure change in the cuff, the indicator including a manually-displaceable member having a portion that can be aligned with the indicator member when the cuff is at the desired pressure so that change in cuff pressure is indicated by movement of the indicator member away from the portion of the manually-displaceable member, wherein the sealing cuff is connected to the cuff pressure indicator such that the cuff pressure indicator provides an indication of pressure change within the sealing cuff.
